(19) United States
(12) Patent Application Publication
(10) Pub. No.: US 2009/0311750 A1
(43) Pub. Date: Dec. 17, 2009

(54) METHODS OF CONVERTING FAB SEQUENCES INTO SINGLE CHAIN ANTIBODY SEQUENCES

(76) Inventor: James W. West, Santa Barbara, CA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/139,417

(22) Filed: Jun. 13, 2008

(51) Int. Cl. C12P 21/04

(52) U.S. Cl. 435/69.6

(57) ABSTRACT

In various embodiments, the present invention provides methods of converting a Fab molecule to a scFv molecule. The methods include amplifying the polynucleotide sequence of the variable light and variable heavy regions of the Fab molecule from the framework 1 to framework 4 sequences; adding one or more restriction endonuclease sites to the amino or carboxyl terminal of the amplified variable region sequence by PCR or ligation; adding one or more linker sequences to the amino or carboxyl terminal of the amplified variable region sequence by PCR or ligation. Additionally, in various embodiments the variable light chain sequence is either amino terminal or carboxyl terminal to the variable heavy chain sequence.

### BACKGROUND OF THE INVENTION

Many antigen binding proteins are supplied in the Fab format. These can be difficult to express as well as undesirable to use as a therapeutic. scFv molecules can be easier to express as well as better therapeutic candidates. Thus, a process for converting DNA encoding variable domains of antibodies or Fabs in such a way as to encode single chain Fvs is needed. A process is provided herein that is based on PCR and is designed to capture not only the diversity of the CDRs but also the diversity within the framework regions. In addition, the process is designed such that the DNA encoding variable heavy domains and the variable light domains from a population of antibody or Fab genes can be recombined in a combinatorial way to form a larger pool of all combinations of heavy and light domains described by the parental pool of genes. Furthermore the heavy and light encoding DNA can be combined in either the light-heavy or the heavy-light configurations.

This application provides methods for converting Fab fragments to scFv fragments.

### DETAILED DESCRIPTION OF THE INVENTION

In the description that follows, a number of terms are used extensively. The following definitions are provided to facilitate understanding of the invention.

As used herein, the term “antibody” or “antibody peptide(s)” refers to an antibody, or a binding fragment thereof that competes with the antibody for specific binding to its target and includes chimeric, humanized, fully human, and bispecific antibodies as well as diabodies, linear antibodies, multivalent or multispecific hybrid antibodies (as described above and in detail in: Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986), which are incorporated herein by reference).

Antibody fragments include, but are not limited to, Fab, Fab′, F(ab)2 , F(ab′) 2, Fv, single-chain antibodies, and minimal binding unites comprising a LCDR1, LCDR2, and LCDR3 of a light chain variable region and a HCDR1, HCDR2, and HCDR3 of a heavy chain variable region.

The term “agonist” refers to any compound including a protein, polypeptide, peptide, antibody, antibody fragment, large molecule, or small molecule (less than 10 kD), that increases the activity, activation or function of another molecule.

The term “bind(ing) of a polypeptide of the invention to a ligand” includes, but is not limited to, the binding of a ligand polypeptide of the present invention to a receptor; the binding of a receptor polypeptide of the present invention to a ligand; the binding of an antibody of the present invention to an antigen or epitope; the binding of an antigen or epitope of the present invention to an antibody; the binding of an antibody of the present invention to an anti-idiotypic antibody; the binding of an anti-idiotypic antibody of the present invention to a ligand; the binding of an anti-idiotypic antibody of the present invention to a receptor; the binding of an anti-anti-idiotypic antibody of the present invention to a ligand, receptor or antibody, etc.

The “valency” of an antibody or fragment or portion thereof is the number of different molecules that it can combine with.

The “specificity” of an antibody or fragment or portion thereof is its ability to distinguish between different antigens.

The term “chimeric antibody” or “chimeric antibodies” refers to antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A typical therapeutic chimeric antibody is thus a hybrid protein comprising the variable or antigen-binding domain from a mouse antibody and the constant domain from a human antibody, although other mammalian species may be used. Specifically, a chimeric antibody is produced by recombinant DNA technology in which all or part of the hinge and constant regions of an immunoglobulin light chain, heavy chain, or both, have been substituted for the corresponding regions from another animal's immunoglobulin light chain or heavy chain. In this way, the antigen-binding portion of the parent monoclonal antibody is grafted onto the backbone of another species' antibody. One approach, described in EP 0239400 to Winter et al. describes the substitution of one species' complementarity determining regions (CDRs) for those of another species, such as substituting the CDRs from human heavy and light chain immunoglobulin variable region domains with CDRs from mouse variable region domains. These altered antibodies may subsequently be combined with human immunoglobulin constant regions to form antibodies that are human except for the substituted murine CDRs which are specific for the antigen. Methods for grafting CDR regions of antibodies may be found, for example in Riechmann et al. (1988) Nature 332:323-327 and Verhoeyen et al. (1988) Science 239:1534-1536.

As used herein, the term “epitope” refers to a site on an antigen recognized by an antibody or an antigen receptor. A T-cell epitope is a short peptide derived from a protein antigen. It binds to an MHC molecule and is recognized by a particular T cell. B-cell epitopes are antigenic determinants recognized by B cells. Antigenic epitopes need not necessarily be immunogenic. Such epitopes can be linear in nature or can be a discontinuous epitope. Thus, as used herein, the term “conformational epitope” refers to a discontinuous epitope formed by a spatial relationship between amino acids of an antigen other than an unbroken series of amino acids.

As used herein, the term “immunoglobulin” refers to a protein consisting of one or more polypeptides substantially encoded by immunoglobulin genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions.

Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes (about 330 amino acids). Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7 (incorporated by reference in its entirety for all purposes).

An immunoglobulin light or heavy chain variable region consists of a “framework” region interrupted by three hypervariable regions. Thus, the term “hypervariable region” refers to the amino acid residues of an antibody which are responsible for antigen binding. The hypervariable region comprises amino acid residues from a “Complementarity Determining Region” or “CDR” (Kabat et al., Sequences of Proteins of Immunological Interest, 5th Ed. Public Health Service, National Institutes of Health, Bethesda, Md. (1991)) and/or those residues from a “hypervariable loop” in the heavy chain variable domain; Chothia and Lesk, 1987, J. Mol. Biol. 196: 901-917) (both of which are incorporated herein by reference). “Framework Region” or “FR” residues are those variable domain residues other than the hypervariable region residues as herein defined. The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

As used herein, the term “human antibody” includes and antibody that has an amino acid sequence of a human immunoglobulin and includes antibodies isolated from human immunoglobulin libraries or from animals transgenic for one or more human immunoglobulin and that do not express endogenous immunoglobulins, as described, for example, by Kucherlapati et al. in U.S. Pat. No. 5,939,598.

As used herein, the terms “single-chain Fv,” “single-chain antibodies,” “Fv” or “scFv” refer to antibody fragments that comprises the variable regions from both the heavy and light chains, but lacks the constant regions or may contain only a portion of the constant region, but within a single polypeptide chain. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041, the disclosures of which are incorporated by reference for any purpose. In specific embodiments, single-chain antibodies can also be bispecific or multispecific and/or humanized.

A “Fab fragment” is comprised of one light chain and the CH1 and variable regions of one heavy chain. The heavy chain of a Fab molecule cannot form a disulfide bond with another heavy chain molecule.

A “Fab′ fragment” contains one light chain and one heavy chain that contains more of the constant region, between the C H1 and C H2domains, such that an interchain disulfide bond can be formed between two heavy chains to form a F(ab′) 2 molecule.

A “F(ab′) 2fragment” contains two light chains and two heavy chains containing a portion of the constant region between the C H1 and C H1domains, such that an interchain disulfide bond is formed between two heavy chains.

The term “diabodies” refers to small antibody fragments with two antigen-binding sites, which fragments comprise a heavy chain variable domain (V H) connected to a light chain variable domain (V L) in the same polypeptide chain (V H—VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The term “linear antibodies” refers to the antibodies described in Zapata et al. Protein Eng. 8(10):1057-1062 (1995). Briefly, these antibodies comprise a pair of tandem Fd segments (VH—CH1-VH—CH1) which form a pair of antigen binding regions. Linear antibodies can be bispecific or monospecific.

The term “immunologically functional immunoglobulin fragment” as used herein refers to a polypeptide fragment that contains at least the variable domains of the immunoglobulin heavy and light chains. An immunologically functional immunoglobulin fragment of the invention is capable of binding to a ligand, preventing binding of the ligand to its receptor, interrupting the biological response resulting from ligand binding to the receptor, or any combination thereof.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

Due to the imprecision of standard analytical methods, molecular weights and lengths of polymers are understood to be approximate values. When such a value is expressed as “about” X or “approximately” X, the stated value of X will be understood to be accurate to ±10%.

The invention is based in part on the discovery of a novel method of converting one or more antibody fragment molecules to one or more single chain antibody molecules. The method can be applied to Fab molecules presented by phage display as well as from other display formats, such as yeast display and bacterial display. The method can be performed on a single Fab molecule or on more than a single Fab molecule. An advantage of the method is that a single process can be used to convert a Fab to a scFv and to change the orientation of the light chain variable region and the heavy chain variable region from their respective orientations in the Fab to a different orientation in the scFv. This single process can be performed on a single Fab molecule or on more than one Fab molecule. The invention also encompasses a method of expediting affinity maturation of the binding regions of the scFv molecule, or fragment thereof, using the conversion method described herein.

The method comprises using DNA amplification methods, such as PCR, to isolate the DNA from the sequence of the variable light region and the variable heavy region from an antibody fragment, such as a Fab molecule, and to clone these regions into a single chain antibody molecule.

Design of the PCR primers is as follows. The human repertoire of antibodies is comprised of variable domains designated variable kappa (VK), variable lambda (VL), or variable heavy (VH). In addition to the variability in the CDRs, antibody genes also display sequence variability in the framework regions (FR). FR1 describes the 5′ end of the DNA encoding each of the variable domains. There are 14 designated FR1s for VK, 23 FR1s for VL, and 8 FR1s for VH. FR4 describes the 3′ end of each of the variable domains. There are 5 FR4s for VK, 3 VRLs for VL and 6 VRLs for VH.

The first round of PCR captures framework and CDR diversity through application of an array of PCR primers that pairs each FR1 primer with each FR4 primer for each of the variable domains. In addition to amplification of the variable domains, the primers also incorporate either a common 5′ GS-linker tag, a common 5′ vector-tag, a common 3′ linker tag, or a common 3′ vector tag. The 5′ vector tag incorporates an ApaLI restriction recognition site and the 3′ vector tag incorporates a NotI restriction recognition site. These sites can then facilitate cloning by ligation of the amplified DNA into expression or phage display vectors. Following this procedure, amplified DNA molecules, regardless of their initial diversity, now have identical 5′ and 3′ sequence tags that can be used as common priming sites in future PCRs. Oligonucleotide primers for amplification of VK, VL or VH domains were designed to capture all published framework diversity and are shown in Tables 1 and 2.

TABLE 1
5′ Oligos

| Variable | | | SEQ |
|---|---|---|---|
| region | Oligonucleotide sequence | OG # | ID NO: |
| VL1 | AAC GCG TAT GCA AGT GCA CAG CAG | zc58137 | 1 |
| | TCT GTG CTG ACT CAG CCA CCC TC | | |
| VL1 | AAC GCG TAT GCA AGT GCA CAG CAG | zc58138 | 2 |
| | TCT GTG TTG ACT CAG CCA CCC TC | | |
| VL1 | AAC GCG TAT GCA AGT GCA CAG CAG | zc58139 | 3 |
| | TCT GTG CTG ACG CAG CCG CCC TC | | |
| VL2 | AAC GCG TAT GCA AGT GCA CAG CAG | zc58171 | 4 |
| | TCT GCC CTG ACT CAG CCT | | |
| VL3 | AAC GCG TAT GCA AGT GCA CAG TCC | zc58172 | 5 |
| | TAT GAG CTG ACT CAG C | | |
| VL3 | AAC GCG TAT GCA AGT GCA CAG TCC | zc58173 | 6 |
| | TAT GAG CTG ACA CAG C | | |
| VL3 | AAC GCG TAT GCA AGT GCA CAG TCT | zc58174 | 7 |
| | TCT GAG CTG ACT CAG G | | |
| VL3 | AAC GCG TAT GCA AGT GCA CAG TCC | zc58175 | 8 |
| | TAT GTG CTG ACT CAG C | | |
| VL3 | AAC GCG TAT GCA AGT GCA CAG TCC | zc58176 | 9 |
| | TAT GAG CTG AAT CAG C | | |
| VL3 | AAC GCG TAT GCA AGT GCA CAG TCC | zc58177 | 10 |
| | TAT GAG CTG ATG CAG C | | |
| VL4 | AAC GCG TAT GCA AGT GCA CAG CTG | zc58178 | 11 |
| | CCT GTG CTG ACT CA | | |
| VL4 | AAC GCG TAT GCA AGT GCA CAG CAG | zc58179 | 12 |
| | CCT GTG CTG ACT CA | | |
| VL4 | AAC GCG TAT GCA AGT GCA CAG CAG | zc58180 | 13 |
| | CTT GTG CTG ACT CA | | |
| VL5 | AAC GCG TAT GCA AGT GCA CAG CAG | zc58181 | 14 |
| | CCT GTG CTG ACT CAG CC | | |
| VL5 | AAC GCG TAT GCA AGT GCA CAG CAG | zc58182 | 15 |
| | GCT GTG CTG ACT CAG CC | | |
| VL6 | AAC GCG TAT GCA AGT GCA CAG AAT | zc58207 | 16 |
| | TTT ATG CTG ACT CAG CCC | | |
| VL7 | AAC GCG TAT GCA AGT GCA CAG CAG | zc58140 | 17 |
| | ACT GTG GTG ACT CAG GAG CCC | | |
| VL7 | AAC GCG TAT GCA AGT GCA CAG CAG | zc58141 | 18 |
| | GCT GTG GTG ACT CAG GAG CCC | | |
| VL8 | AAC GCG TAT GCA AGT GCA CAG CAG | zc58142 | 19 |
| | ACT GTG GTG ACC CAG GAG CC | | |
| VL9 | AAC GCG TAT GCA AGT GCA CAG CAG | zc58143 | 20 |
| | CCT GTG CTG ACT CAG CCA CC | | |
| VL1O | AAC GCG TAT GCA AGT GCA CAG CAG | zc58144 | 21 |
| | GCA GGG CTG ACT CAG CCA CCC | | |
| VLnew1) | AAC GCG TAT GCA AGT GCA CAG CAG | ZC58367 | 22 |
| | TAC GAA TTG ACT CAG CCT GCC | | |
| VLnew2) | AAC gcg tat gca agt gca cag cag age gaa ttg act | zc58594 | 23 |
| | cag cc | | |
| VLnew3) | AAC gcg tat gca agt gca cag TTG ACT CAG | zc58605 | 24 |
| | TCA CCC TCA TTG | | |
| VLnew4) | AAC gcg tat gca agt gca cag TTG ACT CAG | zc58606 | 25 |
| | CCA CCC TCA GTG | | |
| VK1 | AAC GCG TAT GCA AGT GCA CAG RAC | zc57678 | 26 |
| | ATC CAG ATG ACC CAG TCT CC | | |
| VK1 | AAC GCG TAT GCA AGT GCA CAG GMC | zc57762 | 27 |
| | ATC CAG TTG ACC CAG TCT CC | | |
| VK1 | AAC GCG TAT GCA AGT GCA CAG GCC | zc57763 | 28 |
| | ATC YGG ATG ACC CAG TCT CC | | |
| VK1 | AAC GCG TAT GCA AGT GCA CAG GCC | zc57764 | 29 |
| | ATC CAG ATG ACC CAG TCT CC | | |
| VK2 | AAC GCG TAT GCA AGT GCA CAG GAT | zc57765 | 30 |
| | ATT GTG ATG ACC CAG ACT CCA CTC | | |
| VK2 | AAC GCG TAT GCA AGT GCA CAG GAT | zc57766 | 31 |
| | RTT GTG ATG ACT CAG TCT CCA CTC | | |
| VK3 | AAC GCG TAT GCA AGT GCA CAG GAA | zc57767 | 32 |
| | ATT GTG TTG ACR CAG TCT CC | | |
| VK3 | AAC GCG TAT GCA AGT GCA CAG GAA | zc57768 | 33 |
| | ATA GTG ATG ACG CAG TCT CC | | |
| VK3 | AAC GCG TAT GCA AGT GCA CAG GAA | zc57769 | 34 |
| | ATT GTA ATG ACG CAG TCT CC | | |
| VK4 | AAC GCG TAT GCA AGT GCA CAG GAC | zc57770 | 35 |
| | ATC GTG ATG ACC CAG TCT CC | | |
| VK5 | AAC GCG TAT GCA AGT GCA CAG GAA | zc57771 | 36 |
| | ACG ACA CTC ACG CAG TCT CC | | |
| VK6 | AAC GCG TAT GCA AGT GCA CAG GAA | zc57772 | 37 |
| | ATT GTG CTG ACT CAG TCT CC | | |
| VK6 | AAC GCG TAT GCA AGT GCA CAG GAT | zc57773 | 38 |
| | GTT GTG ATG ACA CAG TCT CC | | |
| JK 1) | C ATC AGC CCG AGC GGC CGC AAG TTT | zc57790 | 39 |
| | GAT TTC CAC CTT GGT CCC | | |
| JK 2) | C ATC AGC CCG AGC GGC CGC AAG TTT | zc57791 | 40 |
| | GAT CTC CAG CTT GGT CCC | | |
| JK 3) | C ATC AGC CCG AGC GGC CGC AAG TTT | zc57792 | 41 |
| | GAT ATC CAC TTT GGT CCC | | |
| JK 4) | C ATC AGC CCG AGC GGC CGC AAG TTT | zc57793 | 42 |
| | GAT CTC CAC CTT GGT CCC | | |
| JK 5) | C ATC AGC CCG AGC GGC CGC AAG TTT | zc57668 | 43 |
| | AAT CTC CAG TCG TGT CCC | | |
| JK 6) | C ATC AGC CCG AGC GGC CGC | zc57662 | 44 |
| | AAG TCT AAG CTC CAG TCG TGT CCC | | |
| JK 7) | C ATC AGC CCG AGC GGC CGC AAG | zc58603 | 45 |
| | CTT GAT TTC CAC CTT GGT CCC | | |
| JL | GA AGC ATC AGC CCG AGC GGC CGC | zc58145 | 46 |
| | TAG GAC GGT GAC CTT GGT CCC | | |
| JL | GA AGC ATC AGC CCG AGC GGC CGC | zc58146 | 47 |
| | TAG GAC GGT CAG CTT GGT CCC | | |
| JL | GA AGC ATC AGC CCG AGC GGC CGC | zc58208 | 48 |
| | GAG GAC GGT CAG CTG GGT GCC | | |
| JL | GA AGC ATC AGC CCG AGC GGC CGC | zc58597 | 49 |
| | CAG GAC GGT GAC CTG GGT CCC | | |
| JH | GA AGC ATC AGC CCG AGC GGC CGC | zc58130 | 50 |
| | GCT TGA GAC GGT GAC CAG GGT GCC | | |
| JH | GA AGC ATC AGC CCG AGC GGC CGC | ZC58131 | 51 |
| | GCT TGA GAC AGT GAC CAG GGT GCC | | |
| JH | GA AGC ATC AGC CCG AGC GGC CGC | ZC58132 | 52 |
| | GCT TGA GAC GGT GAC CAT TGT CCC | | |
| JH | GA AGC ATC AGC CCG AGC GGC CGC | ZC57670 | 53 |
| | GCT TGA GAC GGT GAC CAG GGT TCC | | |
| JH | GA AGC ATC AGC CCG AGC GGC CGC | ZC58133 | 54 |
| | GCT TGA GAC GGT GAC CGT GGT CCC | | |
| JH | GA AGC ATC AGC CCG AGC GGC CGC | zc58134 | 55 |
| | GCT TGA GAC GGT GAC CAG GGT CCC | | |

Each sequence in Table 1 begins with an overlap sequence for pARB013 (SEQ ID NO: 56) followed by a sequence for a restriction site, either an ApaL1 site (SEQ ID NO: 57) for the VL, VK, or VH sequences or a sequence for the NotI site (SEQ ID NO: 58) in the JL, JK, and JH sequences. Following this pARB013 sequence and the restriction site is the framework sequence.

In addition to the primers shown in Table 1, the method contemplates the use of a 5′ primer that is designed to be specific to the library that the variable regions are being amplified from. Herein this primer will be referred to as a “Phage/vector specific” oligo.

TABLE 2
3′ Oligos

| | | | |
|---|---|---|---|
| Variable region | Oligonucleotide sequence | OG # | SEQ ID NO |

| | | | |
|---|---|---|---|
| VL1 | TCT GGA GGT TCA GGC GGA CAG TCT GTG | zc58209 | 59 |
| | CTG ACT CAG CCA CCC TC | | |
| VL1 | TCT GGA GGT TCA GGC GGA CAG TCT GTG | zc58210 | 60 |
| | TTG ACT CAG CCA CCC TC | | |
| VL1 | TCT GGA GGT TCA GGC GGA CAG TCT GTG | zc58211 | 61 |
| | CTG ACG CAG CCG CCC TC | | |
| VL2 | TCT GGA GGT TCA GGC GGA CAG TCT GCC | zc58212 | 62 |
| | CTG ACT CAG CCT | | |
| VL3 | TCT GGA GGT TCA GGC GGA TCC TAT GAG | zc58213 | 63 |
| | CTG ACT CAG C | | |
| VL3 | TCT GGA GGT TCA GGC GGA TCC TAT GAG | zc58214 | 64 |
| | CTG ACA CAG C | | |
| VL3 | TCT GGA GGT TCA GGC GGA TCT TCT GAG | zc58215 | 65 |
| | CTG ACT CAG G | | |
| VL3 | TCT GGA GGT TCA GGC GGA TCC TAT GTG | zc58216 | 66 |
| | CTG ACT CAG C | | |
| VL3 | TCT GGA GGT TCA GGC GGA TCC TAT GAG CTG | zc58217 | 67 |
| | AAT CAG C | | |
| VL3 | TCT GGA GGT TCA GGC GGA TCC TAT GAG CTG | zc58218 | 68 |
| | ATG CAG C | | |
| VL4 | TCT GGA GGT TCA GGC GGA CTG CCT GTG CTG | zc58230 | 69 |
| | ACT CA | | |
| VL4 | TCT GGA GGT TCA GGC GGA CAG CCT GTG CTG | zc58231 | 70 |
| | ACT CA | | |
| VL4 | TCT GGA GGT TCA GGC GGA CAG CTT GTG CTG | zc58232 | 71 |
| | ACT CA | | |
| VL5 | TCT GGA GGT TCA GGC GGA CAG CCT GTG CTG | zc58233 | 72 |
| | ACT CAG CC | | |
| VL5 | TCT GGA GGT TCA GGC GGA CAG GCT GTG CT | zc58234 | 73 |
| | ACT CAG CC | | |
| VL6 | TCT GGA GGT TCA GGC GGA AAT TTT ATG CTG | zc58235 | 74 |
| | ACT CAG CCC | | |
| VL7 | TCT GGA GGT TCA GGC GGA CAG ACT GTG GT | zc58236 | 75 |
| | ACT CAG GAG CCC | | |
| VL7 | TCT GGA GGT TCA GGC GGA CAG GCT GTG GT | zc58237 | 76 |
| | ACT CAG GAG CCC | | |
| VL8 | TCT GGA GGT TCA GGC GGA CAG ACT GTG GT | zc58238 | 77 |
| | ACC CAG GAG CC | | |
| VL9 | TCT GGA GGT TCA GGC GGA CAG CCT GTG CTG | zc58239 | 78 |
| | ACT CAG CCA CC | | |
| VL10 | TCT GGA GGT TCA GGC GGA CAG GCA GGG CT | zc58240 | 79 |
| | ACT CAG CCA CCC | | |
| VLnew 1) | TCT GGA GGT TCA GGC GGA CAG TAC GAA TT | ZC58356 | 80 |
| | ACT CAG CCT GCC CCT GCC | | |
| VLnew 2) | TCT GGA GGT TCA GGC GGA CAG AGC GAA TT | zc58595 | 81 |
| | ACT CAG CC CC | | |
| VLnew 3) | TCT GGA GGT TCA GGC GGA TTG ACT CAG | ZC58607 | 82 |
| | TCA CCC TCA TTG | | |
| VLnew 4) | TCT GGA GGT TCA GGC GGA TTG ACT CAG | ZC58608 | 83 |
| | CCA CCC TCA GTG | | |
| VK1 | TCT GGA GGT TCA GGC GGA RAC ATC CAG | zc57679 | 84 |
| | ATG ACC CAG TCT CC | | |
| VK1 | TCT GGA GGT TCA GGC GGA GMC ATC CAG | zc57774 | 85 |
| | TTG ACC CAG TCT CC | | |
| VK1 | TCT GGA GGT TCA GGC GGA GCC ATC YGG | zc57775 | 86 |
| | ATG ACC CAG TCT CC | | |
| VK1 | TCT GGA GGT TCA GGC GGA GCC ATC CAG | zc57776 | 87 |
| | ATG ACC CAG TCT CC | | |
| VK2 | TCT GGA GGT TCA GGC GGA GAT ATT GTG | zc57777 | 88 |
| | ATG ACC CAG ACT CCA CTC | | |
| VK2 | TCT GGA GGT TCA GGC GGA GAT GTT GTG | zc57778 | 89 |
| | ATG ACT CAG TCT CCA CTC | | |
| VK3 | TCT GGA GGT TCA GGC GGA GAA ATT GTG | zc57779 | 90 |
| | TTG ACR CAG TCT CC | | |
| VK3 | TCT GGA GGT TCA GGC GGA GAAATA GTG | zc57780 | 91 |
| | ATG ACG CAG TCT CC | | |
| VK3 | TCT GGA GGT TCA GGC GGA GAA ATT GTA | zc57781 | 92 |
| | ATG ACG CAG TCT CC | | |
| VK4 | TCT GGA GGT TCA GGC GGA GAC ATC GTG | zc57782 | 93 |
| | ATG ACC CAG TCT CC | | |
| VK5 | TCT GGA GGT TCA GGC GGA GAA ACG ACA | zc57783 | 94 |
| | CTC ACG CAG TCT CC | | |
| VK6 | TCT GGA GGT TCA GGC GGA GAA ATT GTG | zc57784 | 95 |
| | CTG ACT CAG TCT CC | | |
| VK6 | TCT GGA GGT TCA GGC GGA GAT GTT GTG | zc57785 | 96 |
| | ATG ACA CAG TCT CC | | |
| JK | GCC TGA ACC TCC AGA ACC AAG TTT GAT TTC | zc57786 | 97 |
| | CAC CTT GGT CCC | | |
| JK | GCC TGA ACC TCC AGA ACC AAG TTT GAT CTC | zc57787 | 98 |
| | CAG CTT GGT CCC | | |
| JK | GCC TGA ACC TCC AGA ACC AAG TTT GAT ATC | zc57788 | 99 |
| | CAC TTT GGT CCC | | |
| JK | GCC TGA ACC TCC AGA ACC AAG TTT GAT CTC | zc57789 | 100 |
| | CAC CTT GGT CCC | | |
| JK | GCC TGA ACC TCC AGA ACC AAG TTT AAT CTC | ZC57667 | 101 |
| | CAG TCG TGT CCC | | |
| JK | GCC TGA ACC TCC AGA ACC AAG TCT AAG CTC | ZC57663 | 102 |
| | CAG TCG TGT CCC | | |
| JK | GCC TGA ACC TCC AGA ACC AAG CTT GAT TT | zc58604 | 103 |
| | CAC CTT GGT CCC | | |
| JK | GCC TGA ACC TCC AGA ACC AAC TAG GAC GG | zc58241 | 104 |
| | GAC CTT GGT CCC | | |
| JK | GCC TGA ACC TCC AGA ACC AAC TAG GAC GG | zc58251 | 105 |
| | CAG CTT GGT CCC | | |
| JK | GCC TGA ACC TCC AGA ACC AAC GAG GAC GG | zc58252 | 106 |
| | CAG CTG GGT GCC | | |
| JK | GCC TGA ACC TCC AGA ACC AAC CAG GAC GG | zc58596 | 107 |
| | GAC CTG GGT CCC | | |
| JH | GCC TGA ACC TCC AGA ACC AAC GCT TGA GA | zc58125 | 108 |
| | GGT GAC CAG GGT GCC | | |
| JH | GCC TGA ACC TCC AGA ACC AAC GCT TGA GA | ZC58126 | 109 |
| | AGT GAC CAG GGT GCC | | |
| JH | GCC TGA ACC TCC AGA ACC AAC GCT TGA GA | ZC58127 | 110 |
| | GGT GAC CAT TGT CCC | | |
| JH | GCC TGA ACC TCC AGA ACC AAC GCT TGA GA | ZC57669 | 111 |
| | GGT GAC CAG GGT TCC | | |
| JH | GCC TGA ACC TCC AGA ACC AAC GCT TGA GA | ZC58128 | 112 |
| | GGT GAC CGT GGT CCC | | |
| JH | GCC TGA ACC TCC AGA ACC AAC GCT TGA GA | ZC58129 | 113 |
| | GGT GAC CAG GGT CCC | | |

Each sequence in Table 2 begins with a GS linker sequence and is followed by a framework sequences.

The second round of PCR lengthens the vector overlaps using the 5′ vector or 3′ vector tag as the priming site, or lengthens the GS linker using the GS-linker tag as the priming site. Oligonucleotide primers for addition of the GS linkers are shown in Table 3.

TABLE 3

| | | | | |
|---|---|---|---|---|
| 3′ or 5′ | GS1 or GS2 | OG sequence | OG3 | SEQ ID NO: |

| | | | | |
|---|---|---|---|---|
| 3′ | GS1 | ACC ACC GCC ACC AGA ACC ACC ACC ACC | zc57674 | 114 |
| | | AGA GCC GCC ACC GCC TGA ACC TCC AGA | | |
| | | ACC AA | | |
| 5′ | GS1 | GGT GGC GGC TCT GGT GGT GGT GGT TCT | zc57675 | 115 |
| | | GGT GGC GGT GGT TCT GGA GGT TCA GGC | | |
| | | GGA | | |
| 3′ | GS2 | ACC GCC ACC GCC AGA ACC GCC CCC GCC | zc57673 | 116 |
| | | AGA ACC GCC GCC GCC TGA ACC TCC AGA | | |
| | | ACC AA | | |
| 5′ | GS2 | GGC GGC GGT TCT GGC GGG GGC GGT TCT | ZC57672 | 117 |
| | | GGC GGT GGC GGT TCT GGA GGT TCA GGC | | |
| | | GGA | | |

Expression plasmid primers are also used. For example, a 5′ recombination oligo of the sequence: GCA TCT ATG TTC GTT TTT TCT ATT GCT ACA AAC GCG TAT GCA AGT GCA CAG (zc57676, SEQ ID NO: 118) was used for expression in vector pARB013. Similarly, a 3′ recombination oligo of the sequence: GAT CAG CTT TTG TTC GGA TCC AGC GGC CGA AGC ATC AGC CCG AGC GGC CGC (zc57981, SEQ ID NO: 119) was used for expression in vector pARB013

The long GS linkers can then be used in round 3 overlap PCR to generate the ScFvs, with long 5′ and 3′ vector overlaps, a 5′ ApaL1 restriction enzyme site and a 3′ NotI restriction enzyme site. The long vector overlaps can then be used to facilitate yeast recombination of the resulting ScFvs into expression or phage display vectors. Following cutting with the restriction enzymes ApaLI and NotI the ScFv encoding DNA can be inserted into expression or phage display vectors by ligation.

Once the conversion process has been performed the scFv molecules can be expressed in a number of hosts and expression systems known in the art. Purification of the scFv proteins is also known in the art, which is complemented by the teachings in Example 3 herein.

Antibody fragments can be produced by recombinant DNA techniques, expression, synthesis, or by enzymatic or chemical cleavage of intact antibodies. Bispecific antibodies may be produced by a variety of methods including, but not limited to, fusion of hybridomas or linking of Fab′ fragments. See, e.g., Songsivilai & Lachmann (1990), Clin. Exp. Immunol. 79:315-321; Kostelny et al. (1992), J. Immunol. 148:1547-1553.

Refolding single chain antibodies (scFV) produced as inclusion bodies in E. coli is known in the art. For example, a rapid and inexpensive refolding method for single chain antibodies is discussed by Sinacola, J. R. and A. S. Robinson, Protein Exp Purif. (26), 301-308, 2002. In this method, anti-fluorescein scFvs are used to study the refolding methods. The scFvs are expressed in E. coli BL21 (DE3) cells transformed with the pET-21a vector containing eithr of the 2 scFv's. Growth was in LB medium at 37° C. with IPTG induction at 1 mM at an OD600 nm of 0.5-0.8. Cells are centrifuged and resuspended in lysis buffer containing lysozyme. The cells are exposed to 3 freeze thaw cycles and DNase added to digest the DNA. The inclusion bodies (IB) are harvested by centrifugation and subjected to washing in buffer. For refolding the IB's are solubilized in 6 M guanidine HCl buffer with B-mercaptoethanol. The solution was incubated overnight at 4° C. The solution was clarified by centrifugation. The solute was then applied to a Sephacryl S-200 gel filtration column and fractions containing the scFv are collected and pooled. Additional B-ME was added to 10 mM to reduce all disulfide bonds. Three different refolding methods are used. The starting concentrations are 0.25 mg/ml ScFv.

Purification can be by dialysis can be performed in a number of different ways. For example, in a stepwise dialysis process approximately 3 ml of the solute is added to a Pierce Dialysis cassette (Slydalyzer) with a 10,000 MWCO membrane. The cassette is equilibrated overnight in solubilization buffer to remove the B-ME. Denaturant is slowly removed by a series of overnight equilibration with buffers of decreasing (by 1 M) guanidine levels. The buffer containing 1 M GuHCl is supplemented with 0.4 M Arginine HCl and oxidized glutathione (GSSG). The refolded protein is removed from the cassette.

Similarly, in a redox dialysis process, approximately 3 ml of the solute is added to a Pierce Dialysis cassette (Slydalyzer) with a 10,000 MWCO membrane. The cassette is equilibrated overnight at 4 degrees C. in 2 exchanges of buffer containing 3 M urea, 1 mM oxidixzed glutathione (GSSG) and 0.1 mM reduced glutathione (GSH), pH 7.9. The redox buffer is reduced by 2 days equilibration with buffer without Urea and the redox agents. The refolded sample is removed from the cassette.

For controlled dilution/filtration, 5.0 ml of the solute is added to a standard ultra filtration cell (Amicon) containing a 10,000 MWCO filter. Cycles of solubilization buffer addition followed by filtration to the original volume are repeated until the reducing agent concentration (B-ME) is reduced 1000 fold. A diafiltration scheme is then applied to reduce the denaturant levels (Guanidine HCl) to 2.0 M. The level is then reduced to 1.0 M GuHCl through constant addition of a refolding buffer containing 800 mM arginine HCl, 200 mM NaCl, Tris buffer and 750 uM GSSG at pH 8.3. The GuHCl levels are reduced to 0.25 M by addition of buffer. The samples is then concentrated via ultrafiltration and washed via dialfiltration to remove the remaining GuHCl.

For on-column refolding, see in general, Kou, Geng et. Al., Protein Exp. Purif. (52), 131-138,2007, In this process the 3″ terminal end of a scFv is ligated to the gene for MHC class 1 molecule recognized peptide from mouse Tyrosine related protein 2 (TRP2). To make the vector the heavy and light chain variable region DNA of PCR products of the VH and VL region are cloned into a pGEM-T vector. The C terminal end of the scFv is fused to the TRP-2 peptide. The cloned DNA is then placed into a pET32a vector which vector is transformed into E. coli BL21 for expression. This vector adds an N terminal His tag to the protein. Cells are grown in LB medium at 37° C. and expression induced with IPTG. Cells expressing inclusion bodies are harvested by centrifugation. The cells are re-suspended in lysis buffer (lysozyme) and sonicated to disrupt the cells. The inclusion body fraction is washed in buffer and is ready for refolding. For the refolding process the inclusion bodies (IB's) are solubilized in 8.0 M urea containing NaCl and imidazole at pH 8.0. The mixture is vortexed and centrifuged to produce the solubilized IB fraction. This solution is applied to a Nickel chelating column equilibrated with 8 M urea, 500 mM NaCl and 5 mM imidazole. The column is washed with the same buffer. The bound protein is refolded on the column by the use of a linear gradient from 8.0 to 0 M urea. The refolded protein is eluted with a buffer containing 500 mM imidazole, 500 mM NaCl, in Tris at pH 8.0.

In another example of scFv expression and refolding, see Wan, L et. al. Protein Exp Purif (48), 307-313; 2006. The cloned DNA is placed into a pQE30 expression vector containing an N-terminal His tag. The vector is transformed into E. coli M15 for expression. The expression is induced by growth in LB medium followed by induction with IPTG. The IB's are harvested by sonication and collected by centrifugation The IB's are washed twice in buffer before solubilization. The IB's are solubilized in 8.0 M Urea at pH 8.0 at 4° C. overnight. The IB solute is loaded onto nickel agarose under denaturing conditions (8 M urea) and equilibrated with the binding buffer. After washing the column, the proteins are eluted in 250 mM imidazole in 8 M urea. Alternatively, the solute is bound onto HiTRap SP XL (cation exchanger) in the presence of 8 M urea. The bound protein is eluted in a gradient off using the same buffer and 0.5 M NaCl.

Another process of dilution refolding uses a Matrix Protein Refolding Guide (Pierce Chemical) to evaluate a number of parameters for protein refolding. The major parameters investigated involved the molarity of arginine HCl (0, 0.4 and 0.8 M) mixed with varying levels of urea (0, 1, and 2 M). The additional variants are the redox ratios of GSH to GSSG. Other components evaluated are NaCl levels, temperature and pH. The solute is diluted into the buffers at 50 ug/ml. Most refolding is done at 4° C. for 18 hours.

The invention is further illustrated by the following non-limiting examples.

### EXAMPLES

### Example 1

### Fab to scFv Conversion by PCR

Table 4 describes the Fab clones selected for binding Target A. Table 5 describes the Fab clones selected for binding Target B. Framework classes are described for both the kappa or lambda (light chain) chains and for the heavy chains. Oligonucleotides, specific for the framework DNA sequences were used to amplify individual domains and to add appropriate sequence tags to the ends of the domains. The sequence tags were used as PCR priming sites for subsequent linker addition and overlap PCR to form ScFvs from the Fabs sequences.

TABLE 4

| | |
|---|---|
| Light chain | Heavy chain |

| Clone | FR1 | FR4 | FR1 | FR4 |
|---|---|---|---|---|
| 1 | VK1.1 | JK5 | Phage/vector specific | JH3 |
| 2 | VK1.1 | JK4 | Phage/vector specific | JH3 |
| 3 | VK1.1 | JK4 | Phage/vector specific | JH5 |
| 4 | VK1.1 | JK5 | Phage/vector specific | JH4 |
| 5 | VK1.1 | JK5 | Phage/vector specific | JH5 |
| 6 | VK1.1 | JK5 | Phage/vector specific | JH4 |
| 7 | VLnew1 | JL1 | Phage/vector specific | JH1 |

TABLE 5

| | |
|---|---|
| Light chain | Heavy chain |

| Clone | FR1 | FR4 | FR1 | FR4 |
|---|---|---|---|---|
| 8 | VK1.1 | JK2 | Phage/vector specific | JH5 |
| 9 | VK1.1 | JK5 | Phage/vector specific | JH5 |
| 10 | VK1.1 | JK5 | Phage/vector specific | JH3 |
| 11 | VK1.1 | JK5 | Phage/vector specific | JH3 |
| 12 | VK1.1 | JK7 | Phage/vector specific | JH4 |
| 13 | VK1.1 | JK1 | Phage/vector specific | JH3 |
| 14 | VLnew3 | JL1 | Phage/vector specific | JH5 |
| 15 | VLnew4 | JL1 | Phage/vector specific | JH6 |

TABLE 6
Oligonucleotide primers for round 1 PCR for conversion of Target B
Fabs to ScFvs

| | PCR1 | PCR2 | | |
|---|---|---|---|---|
| | Light | Light | PCR3 | PCR4 |
| | chain | Chain | Heavy chain | Heavy Chain |
| Clone # | Vect-Link | Link-Vect | Vect-Link | Link-Vect |
| #1 | | | | |
| 5′ Oligo | Zc57678 | Zc57679 | Phage/vector | Phage/vector specific |
| | | | specific | |
| 3′ Oligo | Zc57667 | Zc57668 | Zc58127 | Zc58132 |
| #2 | | | | |
| 5′ Oligo | Zc57678 | Zc57679 | Phage/vector | Phage/vector specific |
| | | | specific | |
| 3′ Oligo | Zc57789 | Zc57793 | Zc58127 | Zc58132 |
| #3 | | | | |
| 5′ Oligo | Zc57678 | Zc57679 | Phage/vector | Phage/vector specific |
| | | | specific | |
| 3′ Oligo | Zc57789 | Zc57793 | Zc58128 | Zc58133 |
| #4 | | | | |
| 5′ Oligo | Zc57678 | Zc57679 | Phage/vector | Phage/vector specific |
| | | | specific | |
| 3′ Oligo | Zc57667 | Zc57668 | Zc57669 | Zc57670 |
| #5 | | | | |
| 5′ Oligo | Zc57678 | Zc57679 | Phage/vector | Phage/vector specific |
| | | | specific | |
| 3′ Oligo | Zc57667 | Zc57668 | Zc58128 | Zc58133 |
| #6 | | | | |
| 5′ Oligo | Zc57678 | Zc57679 | Phage/vector | Phage/vector specific |
| | | | specific | |
| 3′ Oligo | Zc57667 | Zc57668 | Zc57669 | Zc57670 |
| #7 | | | | |
| 5′ Oligo | Zc58367 | Zc58356 | Phage/vector | Phage/vector specific |
| | | | specific | |
| 3′ Oligo | Zc58241 | Zc58145 | Zc57669 | Zc57670 |

TABLE 7
Oligonucleotide primers for round 1 PCR for conversion of anti-Target A
Fabs to ScFvs

| | Round 1 | | | |
|---|---|---|---|---|
| | | PCR2 | | |
| | PCR1 | Light | | |
| | Light | Chain | PCR3 | PCR4 |
| | chain | Link- | Heavy chain | Heavy Chain |
| | Vect-Link | Vect | Vect-Link | Link-Vect |
| #8 | | | | |
| 5′ Oligo | Zc57678 | Zc57679 | Phage/vector | Phage/vector specific |
| | | | specific | |
| 3′ Oligo | Zc57791 | Zc57787 | Zc58128 | Zc58133 |
| #9 | | | | |
| 5′ Oligo | Zc57678 | Zc57679 | Phage/vector | Phage/vector specific |
| | | | specific | |
| 3′ Oligo | Zc57791 | Zc57787 | Zc58128 | Zc58133 |
| #10 | | | | |
| 5′ Oligo | Zc57678 | Zc57679 | Phage/vector | Phage/vector specific |
| | | | specific | |
| 3′ Oligo | Zc57667 | Zc57668 | Zc58127 | Zc58132 |
| #11 | | | | |
| 5′ Oligo | Zc57678 | Zc57679 | Phage/vector | Phage/vector specific |
| | | | specific | |
| 3′ Oligo | Zc57667 | Zc57668 | Zc58127 | Zc58132 |
| #12 | | | | |
| 5′ Oligo | Zc57678 | Zc57679 | Phage/vector | Phage/vector specific |
| | | | specific | |
| 3′ Oligo | Zc58604 | Zc58603 | Zc57669 | Zc57670 |
| #13 | | | | |
| 5′ Oligo | Zc57678 | Zc57679 | Phage/vector | Phage/vector specific |
| | | | specific | |
| 3′ Oligo | Zc57786 | Zc57790 | Zc58127 | Zc58132 |
| #14 | | | | |
| 5′ Oligo | Zc58605 | Zc58607 | Phage/vector | Phage/vector specific |
| | | | specific | |
| 3′ Oligo | Zc58596 | Zc58597 | Zc58128 | Zc58133 |
| #15 | | | | |
| 5′ Oligo | Zc58606 | Zc58608 | Phage/vector | Phage/vector specific |
| | | | specific | |
| 3′ Oligo | Zc58241 | Zc58145 | Zc58129 | Zc58134 |

Each round 1 PCR was carried out using Advantage II polymerase (Clontech, Inc.) with approximately 7 ng template DNA,2 pmoles of each primer, 400 μM dNTPs. Following 10 cycles, all round 1 PCR1 reactions for anti-IL17 templates were pooled. Likewise all PCR2 reactions, PCR3 reactions and PCR4 reactions were pooled. The same was done for anti-IL23 round 1 PCRs. The pooled round 1 PCR reactions were used as templates for round 2 PCRs.

Round 2 PCRs utilize the terminal sequences added during round 1 PCR as priming sites for amplification of the antibody domain sequences. In addition, round 2 PCR adds additional sequence to facilitate either overlap PCR or yeast recombination. For overlap PCR the additional sequence encodes a Gly-Ser repeat linker of 31 amino acids and for yeast recombination the added DNA is complementary to the DNA flanking the insertion site in the expression vector pARB013. In addition, the Gly-Ser linker sequence is different for anti-Target A and anti-Target B, and any linker could be added at this time.

TABLE 8
Oligonucleotide primers for round 2 PCR for conversion of anti-Target
B and anti-Target A Fabs to ScFvs

| | Round 2 | | | |
|---|---|---|---|---|
| | PCR1 | PCR2 | PCR3 | PCR4 |
| Template | Round 1 | Round 1 PCR2 | Round 1 PCR3 | Round 1 |
| anti-Target | PCR1 pool | pool | pool | PCR4 |
| B | | | | pool |
| 5′ oligo | Zc57676 | Zc57675 | Zc57676 | Zc57675 |
| 3′ oligo | Zc57674 | Zc57981 | Zc57674 | Zc57981 |
| Template | Round 1 | Round 1 PCR2 | Round 1 PCR3 | Round 1 |
| anti-Target | PCR1 pool | pool | pool | PCR4 |
| A | | | | pool |
| 5′ oligo | Zc57676 | Zc57672 | Zc57676 | Zc57672 |
| 3′ oligo | Zc57673 | Zc57981 | Zc57673 | Zc57981 |

Each round 3 PCR was carried out using Advantage II polymerase (Clontech, Inc.) with 6 μI of round 1 PCR pools as template DNA,10 pmoles of each primer, and 400 μM dNTPs. Following 10 cycles, PCR products were purified from 1% agarose, TAE gels using Qiagen gel isolation kit as per manufacturer's instructions (Qiagen, Inc.) Round 2 PCR products were then used as templates in round 3 overlap PCR to synthesize ScFvs.

TABLE 9
Oligonucleotide primers for round 3 PCR for conversion of anti-Target
B and anti-Target A Fabs to ScFvs

| | Round 3 | | | |
|---|---|---|---|---|
| | IL23ScFv | IL23 ScFv | IL-17 ScFv | IL-17 ScFv |
| | PCR1 | PCR2 | PCR1 | PCR2 |
| Domain orient | Light-Heavy | Heavy-Light | Light-Heavy | Heavy-Light |
| Template 1 | Round 2 | Round 2 | Round 2 | Round 2 |
| | PCR1 | PCR3 | PCR1 | PCR3 |
| Template 2 | Round 2 | Round 2 | Round 2 | Round 2 |
| | PCR4 | PCR2 | PCR4 | PCR2 |
| 5′ oligo | Zc57676 | Zc57676 | Zc57676 | Zc57676 |
| 3′oligo | Zc57981 | Zc57981 | Zc57981 | Zc57981 |

Expression Constructs

Constructs for the expression of the ScFvs were made in pARB013. pARB013, cut with SmaI, was used in recombination with the PCR insert fragments. Plasmid pARB013 is an E. coli expression vector containing an expression cassette having the PhoA promoter, an E. coli origin of replication; a Kanamycin resistance gene, and URA3 and CEN-ARS sequences required for selection and replication in S. cerevisiae. Following recombination, yeast DNA was recovered and transformed into the E. coli host TG-1. E. coli clones expressing ScFvs were isolated and the purified protein was analyzed for binding to Target A and Target B.

Phage Display

Constructs for phage display of the resulting ScFvs were made in a phage display vector, cut with the restriction enzymes ApaL1 and Not1, and the linear DNA was purified from 1% agarose; TEA gel using qiagen, gel extraction kit as per manufacturer's instructions (Qiagen, Inc.). Round 3 PCR products were cut with the restriction enzymes ApaL1 and Not1, and the DNA was purified from 1% agarose;TEA gel using Qiagen gel extraction kit as per manufacturer's instructions (Qiagen, Inc.). ScFv endoding DNa fragments were inserted into ApaLI/NotI cut vector in a ligation reaction using T4-DNA ligase. Transformation of the ScFv containing vector into th E. coli host TG-1 results in host bacteria capable of producing bacteriophage that display ScFvs on their surface as gene III fusions. DNA sequencing revealed correct conversion of the FABs to ScFvs including shuffling of domains in both the light-heavy and heavy-light orientation.

### Example 2

### Tandem Single Chain Fv Construction from Fabs

The construction of TaScFvs is achieved through three rounds of PCR.

Bispecific molecules can be generated using the following procedure.

In Round four PCR, the tether sequences are used to generate, in a combinatorial way, TaScFvs by overlap PCR which are further amplified using the 3′ and 5′ vector sequences as priming sites. The long vector overlaps can then be used to facilitate yeast recombination of the resulting ScFvs into expression or phage display vectors. Following cutting with the restriction enzymes ApaLI and NotI the TaScFv encoding DNA can be inserted into expression or phage display vectors by ligation.

As Example

Each round 1 PCR was carried out using Advantage II polymerase (Clontech, Inc.) with approximately 7 ng template DNA, 2 pmoles of each primer, 400 μM dNTPs. Following 15 cycles, the PCR products were purified from a 1% AGAROSE:TAE gel using Qiagen gel extraction kit (Qiagen, Inc,) as per manufacturers instructions. Round 1 PCR products were used as template for round 2.

TABLE 10
Round one PCR, Tag addition

| | | | | | | |
|---|---|---|---|---|---|---|
| Round 1 | PCR1 | PCR2 | PCR3 | PCR4 | PCR5 | PCR6 |

A: Anti-Target A

| | | | | | | |
|---|---|---|---|---|---|---|
| Oligo 1 | Zc57678 | Zc57679 | Phage/vector specific | Phage/vector specific | Zc57679 | Phage/vector specific |
| Oligo2 | Zc57663 | Zc57662 | Zc58127 | Zc58132 | Zc57663 | Zc58127 |

B: Anti-Target B

| | | | | | | |
|---|---|---|---|---|---|---|
| Oligo 1 | Zc57678 | Zc57679 | Phage/vector specific | Phage/vector specific | Zc57679 | Phage/vector specific |
| Oligo2 | Zc57667 | Zc57668 | Zc58669 | Zc58670 | Zc57667 | Zc58669 |

Round 2 PCRs utilize the terminal sequences added during round 1 PCR as priming sites for amplification of the antibody domain sequences. Templates and primers are shown in table 7. In addition, round 2 PCR adds additional sequence to facilitate either overlap PCR or yeast recombination. For overlap PCR to form ScFv in round 3 the additional sequence encodes a Gly-Ser repeat linker of 31 amino acids. For overlap PCR to form TAScFv in round 4the additional sequence encodes a tether GSGGSG(EAAAK)4GSGGSG. To facilitate yeast recombination the added DNA is complementary to the DNA flanking the insertion site in the expression vector pARB013. In addition, the DNA encoding the Gly-Ser linker sequence is different for anti-Target A and anti-Target B. Each round 1 PCR was carried out using Advantage II polymerase (Clontech, Inc.) with approximately 7 ng template DNA (Table 11), 10 pmoles of each primer (Table 11), 400 μM dNTPs. Following 15 cycles, the PCR products were purified from a 1% AGAROSE:TAE gel using Qiagen gel extraction kit (Qiagen, Inc,) as per manufacturers instructions. Following 15 cycles, PCR products were purified from 1% agarose, TAE gels using Qiagen gel extraction kit as per manufacturer's instructions (Qiagen, Inc.) Round 2 PCR products were then used as templates in round 3 overlap PCR to synthesize ScFvs. Although specific examples are illustrated here, linker or tether sequences are not limited by the example sequences illustrated here and could comprise any sequence and any linker could be added at this time.

TABLE 11
Round 2 Linker and tether additon
A: Anti-Target A

| Round 2 | PCR1 | PCR2 | PCR3 | PCR4 | PCR5 | PCR6 | PCR7 | PCR8 |
|---|---|---|---|---|---|---|---|---|
| Template | RD1PCR1 | RD1PCR2 | RD1PCR5 | RD1PCR5 | RD1PCR3 | RD1PCR4 | RD1PCR6 | RD1PCR6 |
| Oligo1 | Zc57676 | Zc57675 | Zc57675 | Zc57706 | Zc57676 | Zc57675 | Zc57675 | Zc57706 |
| Oligo2 | Zc57674 | Zc57981 | Zc57705 | Zc557674 | Zc57674 | Zc57981 | Zc57705 | Zc557674 |

B: Anti-Target B

| Round 2 | PCR9 | PCR10 | PCR11 | PCR12 | PCR13 | PCR14 | PCR15 | PCR16 |
|---|---|---|---|---|---|---|---|---|
| Template | RD1PCR1 | RD1PCR2 | RD1PCR5 | RD1PCR5 | RD1PCR3 | RD1PCR4 | RD1PCR6 | RD1PCR6 |
| Oligo1 | Zc57676 | Zc57675 | Zc57675 | Zc57706 | Zc57676 | Zc57675 | Zc57675 | Zc57706 |
| Oligo2 | Zc57674 | Zc57981 | Zc57705 | Zc557674 | Zc57674 | Zc57981 | Zc57705 | Zc557674 |

Each round 3P CR was carried out using Advantage II polymerase (Clontech, Inc.) with 6 μl of each designated round 2 PCR products as DNA templates (Table 8), 10 pmoles of each primer (Table 8), and 400 μM dNTPs. Following 10 cycles, PCR products were purified from 1% agarose, TAE gels using Qiagen gel isolation kit as per manufacturer's instructions (Qiagen, Inc.) Round 2 PCR products were then used as templates in round 3 overlap PCR to synthesize ScFvs.

TABLE 12
Round 3 PCR: Single chain Fv formation

| Round 3 | PCRA | PCRB | PCRE | PCRF | PCRG | PCRH |
|---|---|---|---|---|---|---|
| Anti-Target A | | | | | | |
| Template 1 (RD2 PCR) | PCR1 | PCR5 | PCR1 | PCR4 | PCR5 | PCR8 |
| Template 2 (RD2 PCR) | PCR6 | PCR2 | PCR7 | PCR6 | PCR3 | PCR2 |
| Oligo 1 | Zc57676 | Zc57676 | Zc57676 | Zc57706 | Zc57676 | Zc57706 |
| Oligo2 | Zc57981 | Zc57981 | Zc57705 | Zc57981 | Zc57705 | Zc57981 |

A: Anti-Target B.

| Round 3 | PCRC | PCRD | PCRI | PCRJ | PCRK | PCRL |
|---|---|---|---|---|---|---|
| Anti-Target B | | | | | | |
| Template 1 (RD2 PCR) | PCR9 | PCR13 | PCR9 | PCR12 | PCR13 | PCR16 |
| Template 2 (RD2 PCR) | PCR14 | PCR10 | PCR15 | PCR14 | PCR11 | PCR10 |
| Oligo 1 | Zc57676 | Zc57676 | Zc57676 | Zc57706 | Zc57676 | Zc57706 |
| Oligo2 | Zc57981 | Zc57981 | Zc57705 | Zc57981 | Zc57705 | Zc57981 |

Each round 4 PCR was carried out using Advantage II polymerase (Clontech, Inc.) with 6 □l of each designated round 3 PCR products as DNA templates (Table 9), 10 pmoles of each primer (Table 9), and 400 μM dNTPs. Following 20 cycles, PCR products were purified from 1% agarose, TAE gels using Qiagen gel isolation kit as per manufacturer's instructions (Qiagen, Inc.).

TABLE 13
Tandem single chain overlap PCR

| Round4 | PCREJ | PCREL | PCRGJ | PCRGL | PCRIF | PCRIH | PCRKF | PCRKH |
|---|---|---|---|---|---|---|---|---|
| Template 1 (RD3 PCR) | PCRE | PCRE | PCRG | PCRG | PCRI | PCRI | PCRK | PCRK |
| Template 2 (RD3 PCR) | PCRJ | PCRL | PCRJ | PCRL | PCRF | PCRH | PCRF | PCRH |
| Oligo 1 | Zc57676 | Zc57676 | Zc57676 | Zc57676 | Zc57676 | Zc57676 | Zc57676 | Zc57676 |
| Oligo2 | Zc57981 | Zc57981 | Zc57981 | Zc57981 | Zc57981 | Zc57981 | Zc57981 | Zc57981 |

Each round 4 PCR was carried out using Advantage II polymerase (Clontech, Inc.) with 6 μl of each designated round 3 PCR products as DNA templates (Table 9), 10 pmoles of each primer (Table 9), and 400 μM dNTPs. Following 20 cycles, PCR products were purified from 1% agarose, TAE gels using Qiagen gel isolation kit as per manufacturer's instructions (Qiagen, Inc.).

Expression Constructs

Constructs for the expression of the ScFvs were made in pARB013. pARB013, cut with SmaI, was used in recombination with the PCR insert fragments. Plasmid pARB013 is an E. coli expression vector containing an expression cassette having the PhoA promoter, an E. coli origin of replication; a Kanamycin resistance gene, and URA3 and CEN-ARS sequences required for selection and replication in S. cerevisiae. Following recombination of purified round 4 PCR products into SmaI cut pARB013, yeast DNA was recovered and transformed into the E. coli host TG-1. E. coli clones expressing three of the possible TaSCFvs (Table 10) were isolated and the purified protein was analyzed for binding to Target A and Target B

### Example 3

### Purification of scFv Proteins Derived from E. coli Periplasmic Fraction

This method pertains to the purification of scFv, tandem scFv, and sFab proteins expressed in the periplasmic space of E. coli cells. The method applies to the ZGOLD5, BL21, BL21*, and TG1 hosts. Scale of ferment ranges from 25 mL shake flask cultures to 2 L batch fed systems.

Periplasmic Fractionation of E. coli Cells

E. coli cells delivered fresh as a spun pellet on ice by the expression group. Wet cell pellet completely re-suspended in periplasting buffer [0.2M Tris, 20% (w/v) sucrose, Complete EDTA-free protease inhibitor cocktail (Roche) pH 7.5] at a ratio of 2 mL per gram of wet cell weight. Lysozyme is an enzyme that facilitates the degradation of the cell wall and may or may not be included in the procedure. To determine whether or not to use lysozyme, 500 uL of re-suspended pellet is transferred to an eppendorf tube and 30 U of Ready-Lyse lysozyme (Epicentre) per uL of periplasting buffer used is added and the suspension incubated at room temperature for 5 minutes. After the incubation, the solution is checked for increased viscosity by inversion. If the solution clings to the wall of the tube, then premature cell lysis may be occurring, and the lysozyme is not included in the preparative solution. If the solution does not cling to the tube wall, then the lysozyme is include in the preparative solution. If using lysozyme, 30 U of Ready-Lyse lysozyme (Epicentre) per uL of periplasting buffer used is added and the suspension incubated at room temperature for 4-6 minutes. Make sure not to incubate for longer than 6 minutes as the enzyme will degrade the cell wall completely, causing cytoplasmic proteins to contaminate the periplasmic prep. Ice cold water is added at a ratio of 3 mL per gram of original wet cell pellet weight and the solution incubated for at least 10 minutes but no longer than 30 minutes. The remaining spheroplasts are pelleted via centrifugation at 15,000×g (or 10,000-20,000 RPM, whichever is faster) for at least 15 minutes, but no longer than 45 minutes, at room temperature. The supernatant containing the periplasmic fraction is poured into a new vessel and adjusted to 25 mM Imidazole, 500 mM NaCl using weighed out solid. This solution is 0.22 um filtered prior to purification using a bottle top filter (Nalgene).

Immobilized Metal Affinity Chromatography (IMAC) Capture

Traditionally, a 5 mL HisTrap HP column (GE Healthcare) is used for the IMAC step, however, the column size can be scaled up or down depending on the amount of scFv target in the periplasmic fraction as determined by an analytical IMAC-SEC assay. Binding capacity of this IMAC resin has been shown to be at least 20 mg/mL of packed bed. If using columns larger than 10 mL in size, Waters Glass Columns (Millipore) with a 2 and 5 cm internal diameter are preferred. Using an appropriate chromatography station (Akta Explorer using UNICORN software 4.1 and higher [GE Healthcare] or BioCAD Sprint, 700E, or Vision using Perfusion Chromatography software version 3.00 or higher [Applied Biosystems]), the IMAC column is equilibrated in 50 mM NaPO4, 500 mM NaCl, 25 mM Imidazole pH 7.5 and the periplasmic fraction loaded over it at no faster than 190 cm/hr until depleted. Column washed with equilibration buffer until monitors at UV A254 nm and UV A280 nm are baseline stable for at least 2 CV at a flow rate not to exceed 190 cm/hr. Bound protein eluted competitively using 50 mM NaPO4, 500 mM NaCl, 400 mM Imidazole, pH 7.5 at no faster than 190 cm/hr. Elution fractions assessed for protein content via UV @ A280 nm, analytical size exclusion chromatography, and SDS-PAGE.

Other Chormatographic Techniques:

Purity of the IMAC pool is assessed by SDS-PAGE gel and analytical size exclusion chromatography (SEC). If the pool is not amenable to final clean up via SEC, other chromatographic techniques can be employed to further purify the target scFv protein from residual host cell contaminants and aggregates. These conventional techniques may include, but are not limited to: anion exchange, cation exchange, and hydrophobic interaction. Other affinity based approaches can also be used, including, but not limited to: utilization of the c-terminal myc tag via anti-myc resin or ligand based affinity approaches using the appropriate ligand covalently coupled to a rigid bead. The utility of these other techniques are determined on a protein to protein basis.

Size Exclusion Chromatography (SEC)

Amount of protein as assessed by UV @ A280 nm and analytical SEC method determines the size of gel filtration column used: <1 mg=10/300 Superdex 200 GL column, 1-10 mg=16/60 Superdex 200, >10 mg=26/60 Superdex 200 (All GE Healthcare). IMAC elution pool concentrated using 10 kD MWCO Ultracel centrifugal concentrator (Millipore) with the final concentrate volume being no more than 3% of the volume of gel filtration column used. Concentrate injected onto column and the protein eluted isocratically at a flow rate not to exceed 76 cm/hr and no slower than 34 cm/hr. Elution fractions taken, analyzed by SDS-PAGE, and the appropriate pool made.

Endotoxin Removal

Final product specifications regarding endotoxin levels are determined by status of a particular cluster. For proteins destined for later stage usage, the endotoxin removal step is performed. SEC pool concentrated to >0.25 mg/mL as determined by UV @ A280 nm using a 10 kD MWCO Ultracel centrifugal concentrator (Millipore). A Mustang E 0.22 um filter (PALL) pre-wetted with SEC mobile phase buffer and the SEC concentrate filtered through it via manual syringe delivery system at a flow rate of ˜1 mL/min. Final filtered product assayed for endotoxin using PTS EndoSafe system (Charles River), concentration via UV @ A280 nm, and handed to the associate in charge of aliquotting and storage. An acceptable endotoxin limit is 1 EU/mg of purified product. Multiple filtration steps may be needed to achieve this level depending on the amount of endotoxin in the original sample.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of converting a Fab molecule to a scFv molecule comprising the steps of:
a) amplifying the polynucleotide sequence of the variable light and variable heavy regions of the Fab molecule from the framework 1 to framework 4 sequences;
b) adding one or more restriction endonuclease sites to the amino or carboxyl terminal of the amplified variable region sequence by PCR or ligation;
c) adding one or more linker sequences to the amino or carboxyl terminal of the amplified variable region sequence by PCR or ligation;
wherein the resulting PCR product comprises the variable light and variable heavy chains of the Fab molecule in a single chain comprising a linker sequence between the variable light and variable heavy chain sequences and wherein the variable light chain sequence is either amino terminal or carboxyl terminal to the variable heavy chain sequence.

2. The method of claim 1, wherein the restriction endonuclease sites are ApaL1 and Not1 .

3. The method of claim 1, wherein the linkers are comprise a combination of glycine and serine residues.

4. The method of claim 1, wherein the one or more restriction endonuclease sites are added by PCR and the one or more linker sequences to the amino or carboxyl terminal of the amplified variable region sequence are added by PCR.

5. The method of claim 1, comprising a step of displaying the scFv molecule on a host selected from the group selected from the group selected from:
a) phage display;
b) yeast display; and
c) bacterial display.

6. The method of claim 1 wherein the process is performed on a single Fab molecule.

7. The method of claim 1 wherein the process is performed on more than one Fab molecule.

8. The method of claim 1 wherein the resulting scFv comprises a light chain variable region from a different Fab than the Fab that the heavy chain variable region originated from.

9. The method of claim 1, wherein orientation of the light chain variable region and the heavy chain variable region change from their respective orientations in the Fab to a different orientation in the scFv.

10. A method of affinity maturing the binding regions of one or more Fab molecules in conjunction with converting the Fab molecule to a scFv molecule by using a pool of PCR products of variable light and variable heavy regions wherein the method comprises the steps of:
a) amplifying the polynucleotide sequence of the variable light and variable heavy regions of the Fab molecule from the framework 1 to framework 4 sequences;
b) adding one or more restriction endonuclease sites to the amino or carboxyl terminal of the amplified variable region sequence by PCR or ligation;
c) adding one or more linker sequences to the amino or carboxyl terminal of the amplified variable region sequence by PCR or ligation;
wherein the resulting PCR product comprises the variable light and variable heavy chains of the Fab molecule in a single chain comprising a linker sequence between the variable light and variable heavy chain sequences and wherein the variable light chain sequence is either amino terminal or carboxyl terminal to the variable heavy chain sequence.

11. The method of claim 4 wherein the amino terminal primer for the PCR comprises an amino acid sequence selected from the group consisting of:
A) SEQ ID NO: 1
B) SEQ ID NO: 2
C) SEQ ID NO: 3
D) SEQ ID NO:4
E) SEQ ID NO: 5
F) SEQ ID NO: 6
G) SEQ ID NO:7
H) SEQ ID NO:8
I) SEQ ID NO:9
J) SEQ ID NO:10
K) SEQ ID NO:11
L) SEQ ID NO:12
M) SEQ ID NO:13
N) SEQ ID NO:14
O) SEQ ID NO:15
P) SEQ ID NO:16
Q) SEQ ID NO:17
R) SEQ ID NO:18
S) SEQ ID NO:19
T) SEQ ID NO:20
U) SEQ ID NO:21
V) SEQ ID NO:22
W) SEQ ID NO:23
Z) SEQ ID NO:24
Y) SEQ ID NO:25
Z) SEQ ID NO:26
Aa) SEQ ID NO:27
Bb) SEQ ID NO:28
Cc) SEQ ID NO:29
Dd) SEQ ID NO:30
Ee) SEQ ID NO:31
Ff) SEQ ID NO:32
Gg) SEQ ID NO:33
Hh) SEQ ID NO:34
Ii) SEQ ID NO:35
Jj) SEQ ID NO:36
Kk) SEQ ID NO:37
Ll) SEQ ID NO:38
Mm) SEQ ID NO:39
Nn) SEQ ID NO:40
Oo) SEQ ID NO:41
Pp) SEQ ID NO:42
Qq) SEQ ID NO:43
Rr) SEQ ID NO:44
§) SEQ ID NO:45
Tt) SEQ ID NO:46
Uu) SEQ ID NO:47
Vv) SEQ ID NO:48
Ww) SEQ ID NO:49
Xx) SEQ ID NO:50
Yy) SEQ ID NO:51
Zz) SEQ ID NO:52
Aaa) SEQ ID NO:53
Bbb) SEQ ID NO:54
Ccc) SEQ ID NO:55

12. The method of claim 4 wherein the carboxyl terminal primer for the PCR comprises an amino acid sequence selected from the group consisting of:
A) SEQ ID NO:59
B) SEQ ID NO:60
C) SEQ ID NO:61
D) SEQ ID NO:62
E) SEQ ID NO:63
F) SEQ ID NO:64
G) SEQ ID NO:65
H) SEQ ID NO:66
I) SEQ ID NO:67
H) SEQ ID NO:68
K) SEQ ID NO:69
L) SEQ ID NO:70
M) SEQ ID NO:71
N) SEQ ID NO:72
O) SEQ ID NO:73
P) SEQ ID NO:74
Q) SEQ ID NO:75
R) SEQ ID NO:76
S) SEQ ID NO:77
T) SEQ ID NO:78
U) SEQ ID NO:79
V) SEQ ID NO:80
W) SEQ ID NO:81
X) SEQ ID NO:82
Y) SEQ ID NO:83
Z) SEQ ID NO:84
Aa) SEQ ID NO:85
Bb) SEQ ID NO:86
Cc) SEQ ID NO:87
Dd) SEQ ID NO:88
Ee) SEQ ID NO:89
Ff) SEQ ID NO:90
Gg) SEQ ID NO:91
Hh) SEQ ID NO:92
Ii) SEQ ID NO:93
Jj) SEQ ID NO:94
Kk) SEQ ID NO:95
Ll) SEQ ID NO:96
Mm) SEQ ID NO:97
Nn) SEQ ID NO:98
Oo) SEQ ID NO:99
Pp) SEQ ID NO:100
Qq) SEQ ID NO:101
Rr) SEQ ID NO:102
§) SEQ ID NO:103
Tt) SEQ ID NO:104
Uu) SEQ ID NO:105
Vv) SEQ ID NO:106
Ww) SEQ ID NO:107
Xx) SEQ ID NO:108
YY) SEQ ID NO:109
ZZ) SEQ ID NO:110
AAA) SEQ ID NO:111
BBB) SEQ ID NO:112
CCC) SEQ ID NO:113

13. The method of claim 11 wherein the amino terminal primer for the PCR comprises an amino acid sequence selected from the group consisting of:
A) SEQ ID NO:59
B) SEQ ID NO:60
C) SEQ ID NO:61
D) SEQ ID NO:62
E) SEQ ID NO:63
F) SEQ ID NO:64
G) SEQ ID NO:65
H) SEQ ID NO:66
I) SEQ ID NO:67
H) SEQ ID NO:68
K) SEQ ID NO:69
L) SEQ ID NO:70
M) SEQ ID NO:71
N) SEQ ID NO:72
O) SEQ ID NO:73
P) SEQ ID NO:74
Q) SEQ ID NO:75
R) SEQ ID NO:76
S) SEQ ID NO:77
T) SEQ ID NO:78
U) SEQ ID NO:79
V) SEQ ID NO:80
W) SEQ ID NO:81
X) SEQ ID NO:82
Y) SEQ ID NO:83
Z) SEQ ID NO:84
Aa) SEQ ID NO:85
Bb) SEQ ID NO:86
Cc) SEQ ID NO:87
Dd) SEQ ID NO:88
Ee) SEQ ID NO:89
Ff) SEQ ID NO:90
Gg) SEQ ID NO:91
Hh) SEQ ID NO:92
Ii) SEQ ID NO:93
Jj) SEQ ID NO:94
Kk) SEQ ID NO:95
Ll) SEQ ID NO:96
Mm) SEQ ID NO:97
Nn) SEQ ID NO:98
Oo) SEQ ID NO:99
Pp) SEQ ID NO:100
Qq) SEQ ID NO:101
Rr) SEQ ID NO:102
§) SEQ ID NO:103
Tt) SEQ ID NO:104
Uu) SEQ ID NO:105
Vv) SEQ ID NO:106
Ww) SEQ ID NO:107
Xx) SEQ ID NO:108
YY) SEQ ID NO:109
ZZ) SEQ ID NO:110
AAA) SEQ ID NO:111
BBB) SEQ ID NO:112
CCC) SEQ ID NO:113

14. The method of claim 12 wherein the amino terminal primer for the PCR comprises an amino acid sequence selected from the group consisting of:
A) SEQ ID NO: 1
B) SEQ ID NO: 2
C) SEQ ID NO: 3
D) SEQ ID NO:4
E) SEQ ID NO: 5
F) SEQ ID NO: 6
G) SEQ ID NO:7
H) SEQ ID NO:8
I) SEQ ID NO:9
J) SEQ ID NO:10
K) SEQ ID NO:11
L) SEQ ID NO:12
M) SEQ ID NO:13
N) SEQ ID NO:14
O) SEQ ID NO:15
P) SEQ ID NO:16
Q) SEQ ID NO:17
R) SEQ ID NO:18
S) SEQ ID NO:19
T) SEQ ID NO:20
U) SEQ ID NO:21
V) SEQ ID NO:22
W) SEQ ID NO:23
Z) SEQ ID NO:24
Y) SEQ ID NO:25
Z) SEQ ID NO:26
Aa) SEQ ID NO:27
Bb) SEQ ID NO:28
Cc) SEQ ID NO:29
Dd) SEQ ID NO:30
Ee) SEQ ID NO:31
Ff) SEQ ID NO:32
Gg) SEQ ID NO:33
Hh) SEQ ID NO:34
Ii) SEQ ID NO:35
Jj) SEQ ID NO:36
Kk) SEQ ID NO:37
Ll) SEQ ID NO:38
Mm) SEQ ID NO:39
Nn) SEQ ID NO:40
Oo) SEQ ID NO:41
Pp) SEQ ID NO:42
Qq) SEQ ID NO:43
Rr) SEQ ID NO:44
§) SEQ ID NO:45
Tt) SEQ ID NO:46
Uu) SEQ ID NO:47
Vv) SEQ ID NO:48
Ww) SEQ ID NO:49
Xx) SEQ ID NO:50
Yy) SEQ ID NO:51
Zz) SEQ ID NO:52
Aaa) SEQ ID NO:53
Bbb) SEQ ID NO:54
Ccc) SEQ ID NO:55

15. The method of claim 1 wherein the sequence of the linker primer is selected from the group consisting of:
a) SEQ ID NO:114
b) SEQ ID NO:115
c) SEQ ID NO:116
D) SEQ ID NO:117.

16. The method of claim 11 wherein the sequence of the linker primer is selected from the group consisting of:
a) SEQ ID NO:114
b) SEQ ID NO:115
c) SEQ ID NO:116
D) SEQ ID NO:117.

17. The method of claim 12 wherein the sequence of the linker primer is selected from the group consisting of:
a) SEQ ID NO:114
b) SEQ ID NO:115
c) SEQ ID NO:116
D) SEQ ID NO:117.


SEQUENCE LISTING (119 sequences)

SEQ ID NO: 1 - DNA, 47 bp, Homo sapiens
aacgcgtatgcaagtgcacagcagtctgtgctgactcagccaccctc

SEQ ID NO: 2 - DNA, 47 bp, Homo sapiens
aacgcgtatgcaagtgcacagcagtctgtgttgactcagccaccctc

SEQ ID NO: 3 - DNA, 47 bp, Homo sapiens
aacgcgtatgcaagtgcacagcagtctgtgctgacgcagccgccctc

SEQ ID NO: 4 - DNA, 42 bp, Homo sapiens
aacgcgtatgcaagtgcacagcagtctgccctgactcagcct

SEQ ID NO: 5 - DNA, 40 bp, Homo sapiens
aacgcgtatgcaagtgcacagtcctatgagctgactcagc

SEQ ID NO: 6 - DNA, 40 bp, Homo sapiens
aacgcgtatgcaagtgcacagtcctatgagctgacacagc

SEQ ID NO: 7 - DNA, 40 bp, Homo sapiens
aacgcgtatgcaagtgcacagtcttctgagctgactcagg

SEQ ID NO: 8 - DNA, 40 bp, Homo sapiens
aacgcgtatgcaagtgcacagtcctatgtgctgactcagc

SEQ ID NO: 9 - DNA, 40 bp, Homo sapiens
aacgcgtatgcaagtgcacagtcctatgagctgaatcagc

SEQ ID NO: 10 - DNA, 40 bp, Homo sapiens
aacgcgtatgcaagtgcacagtcctatgagctgatgcagc

SEQ ID NO: 11 - DNA, 38 bp, Homo sapiens
aacgcgtatgcaagtgcacagctgcctgtgctgactca

SEQ ID NO: 12 - DNA, 38 bp, Homo sapiens
aacgcgtatgcaagtgcacagcagcctgtgctgactca

SEQ ID NO: 13 - DNA, 38 bp, Homo sapiens
aacgcgtatgcaagtgcacagcagcttgtgctgactca

SEQ ID NO: 14 - DNA, 41 bp, Homo sapiens
aacgcgtatgcaagtgcacagcagcctgtgctgactcagcc

SEQ ID NO: 15 - DNA, 41 bp, Homo sapiens
aacgcgtatgcaagtgcacagcaggctgtgctgactcagcc

SEQ ID NO: 16 - DNA, 42 bp, Homo sapiens
aacgcgtatgcaagtgcacagaattttatgctgactcagccc

SEQ ID NO: 17 - DNA, 45 bp, Homo sapiens
aacgcgtatgcaagtgcacagcagactgtggtgactcaggagccc

SEQ ID NO: 18 - DNA, 45 bp, Homo sapiens
aacgcgtatgcaagtgcacagcaggctgtggtgactcaggagccc

SEQ ID NO: 19 - DNA, 44 bp, Homo sapiens
aacgcgtatgcaagtgcacagcagactgtggtgacccaggagcc

SEQ ID NO: 20 - DNA, 44 bp, Homo sapiens
aacgcgtatgcaagtgcacagcagcctgtgctgactcagccacc

SEQ ID NO: 21 - DNA, 45 bp, Homo sapiens
aacgcgtatgcaagtgcacagcaggcagggctgactcagccaccc

SEQ ID NO: 22 - DNA, 45 bp, Homo sapiens
aacgcgtatgcaagtgcacagcagtacgaattgactcagcctgcc

SEQ ID NO: 23 - DNA, 41 bp, Homo sapiens
aacgcgtatgcaagtgcacagcagagcgaattgactcagcc

SEQ ID NO: 24 - DNA, 42 bp, Homo sapiens
aacgcgtatgcaagtgcacagttgactcagtcaccctcattg

SEQ ID NO: 25 - DNA, 42 bp, Homo sapiens
aacgcgtatgcaagtgcacagttgactcagccaccctcagtg

SEQ ID NO: 26 - DNA, 44 bp, Homo sapiens
aacgcgtatgcaagtgcacagracatccagatgacccagtctcc

SEQ ID NO: 27 - DNA, 44 bp, Homo sapiens
aacgcgtatgcaagtgcacaggmcatccagttgacccagtctcc

SEQ ID NO: 28 - DNA, 44 bp, Homo sapiens
aacgcgtatgcaagtgcacaggccatcyggatgacccagtctcc

SEQ ID NO: 29 - DNA, 44 bp, Homo sapiens
aacgcgtatgcaagtgcacaggccatccagatgacccagtctcc

SEQ ID NO: 30 - DNA, 48 bp, Homo sapiens
aacgcgtatgcaagtgcacaggatattgtgatgacccagactccactc

SEQ ID NO: 31 - DNA, 48 bp, Homo sapiens
aacgcgtatgcaagtgcacaggatrttgtgatgactcagtctccactc

SEQ ID NO: 32 - DNA, 44 bp, Homo sapiens
aacgcgtatgcaagtgcacaggaaattgtgttgacrcagtctcc

SEQ ID NO: 33 - DNA, 44 bp, Homo sapiens
aacgcgtatgcaagtgcacaggaaatagtgatgacgcagtctcc

SEQ ID NO: 34 - DNA, 44 bp, Homo sapiens
aacgcgtatgcaagtgcacaggaaattgtaatgacgcagtctcc

SEQ ID NO: 35 - DNA, 44 bp, Homo sapiens
aacgcgtatgcaagtgcacaggacatcgtgatgacccagtctcc

SEQ ID NO: 36 - DNA, 44 bp, Homo sapiens
aacgcgtatgcaagtgcacaggaaacgacactcacgcagtctcc

SEQ ID NO: 37 - DNA, 44 bp, Homo sapiens
aacgcgtatgcaagtgcacaggaaattgtgctgactcagtctcc

SEQ ID NO: 38 - DNA, 44 bp, Homo sapiens
aacgcgtatgcaagtgcacaggatgttgtgatgacacagtctcc

SEQ ID NO: 39 - DNA, 43 bp, Homo sapiens
catcagcccgagcggccgcaagtttgatttccaccttggtccc

SEQ ID NO: 40 - DNA, 43 bp, Homo sapiens
catcagcccgagcggccgcaagtttgatctccagcttggtccc

SEQ ID NO: 41 - DNA, 43 bp, Homo sapiens
catcagcccgagcggccgcaagtttgatatccactttggtccc

SEQ ID NO: 42 - DNA, 43 bp, Homo sapiens
catcagcccgagcggccgcaagtttgatctccaccttggtccc

SEQ ID NO: 43 - DNA, 43 bp, Homo sapiens
catcagcccgagcggccgcaagtttaatctccagtcgtgtccc

SEQ ID NO: 44 - DNA, 43 bp, Homo sapiens
catcagcccgagcggccgcaagtctaagctccagtcgtgtccc

SEQ ID NO: 45 - DNA, 43 bp, Homo sapiens
catcagcccgagcggccgcaagcttgatttccaccttggtccc

SEQ ID NO: 46 - DNA, 44 bp, Homo sapiens
gaagcatcagcccgagcggccgctaggacggtgaccttggtccc

SEQ ID NO: 47 - DNA, 44 bp, Homo sapiens
gaagcatcagcccgagcggccgctaggacggtcagcttggtccc

SEQ ID NO: 48 - DNA, 44 bp, Homo sapiens
gaagcatcagcccgagcggccgcgaggacggtcagctgggtgcc

SEQ ID NO: 49 - DNA, 44 bp, Homo sapiens
gaagcatcagcccgagcggccgccaggacggtgacctgggtccc

SEQ ID NO: 50 - DNA, 47 bp, Homo sapiens
gaagcatcagcccgagcggccgcgcttgagacggtgaccagggtgcc

SEQ ID NO: 51 - DNA, 47 bp, Homo sapiens
gaagcatcagcccgagcggccgcgcttgagacagtgaccagggtgcc

SEQ ID NO: 52 - DNA, 47 bp, Homo sapiens
gaagcatcagcccgagcggccgcgcttgagacggtgaccattgtccc

SEQ ID NO: 53 - DNA, 47 bp, Homo sapiens
gaagcatcagcccgagcggccgcgcttgagacggtgaccagggttcc

SEQ ID NO: 54 - DNA, 47 bp, Homo sapiens
gaagcatcagcccgagcggccgcgcttgagacggtgaccgtggtccc

SEQ ID NO: 55 - DNA, 47 bp, Homo sapiens
gaagcatcagcccgagcggccgcgcttgagacggtgaccagggtccc

SEQ ID NO: 56 - DNA, 12 bp, Homo sapiens
aacgcgtatgca

SEQ ID NO: 57 - DNA, 9 bp, Homo sapiens
agtgcacag

SEQ ID NO: 58 - DNA, 11 bp, Homo sapiens
agcggccgcaa

SEQ ID NO: 59 - DNA, 44 bp, Homo sapiens
tctggaggttcaggcggacagtctgtgctgactcagccaccctc

SEQ ID NO: 60 - DNA, 44 bp, Homo sapiens
tctggaggttcaggcggacagtctgtgttgactcagccaccctc

SEQ ID NO: 61 - DNA, 44 bp, Homo sapiens
tctggaggttcaggcggacagtctgtgctgacgcagccgccctc

SEQ ID NO: 62 - DNA, 39 bp, Homo sapiens
tctggaggttcaggcggacagtctgccctgactcagcct

SEQ ID NO: 63 - DNA, 37 bp, Homo sapiens
tctggaggttcaggcggatcctatgagctgactcagc

SEQ ID NO: 64 - DNA, 37 bp, Homo sapiens
tctggaggttcaggcggatcctatgagctgacacagc

SEQ ID NO: 65 - DNA, 37 bp, Homo sapiens
tctggaggttcaggcggatcttctgagctgactcagg

SEQ ID NO: 66 - DNA, 37 bp, Homo sapiens
tctggaggttcaggcggatcctatgtgctgactcagc

SEQ ID NO: 67 - DNA, 37 bp, Homo sapiens
tctggaggttcaggcggatcctatgagctgaatcagc

SEQ ID NO: 68 - DNA, 37 bp, Homo sapiens
tctggaggttcaggcggatcctatgagctgatgcagc

SEQ ID NO: 69 - DNA, 35 bp, Homo sapiens
tctggaggttcaggcggactgcctgtgctgactca

SEQ ID NO: 70 - DNA, 35 bp, Homo sapiens
tctggaggttcaggcggacagcctgtgctgactca

SEQ ID NO: 71 - DNA, 35 bp, Homo sapiens
tctggaggttcaggcggacagcttgtgctgactca

SEQ ID NO: 72 - DNA, 38 bp, Homo sapiens
tctggaggttcaggcggacagcctgtgctgactcagcc

SEQ ID NO: 73 - DNA, 38 bp, Homo sapiens
tctggaggttcaggcggacaggctgtgctgactcagcc

SEQ ID NO: 74 - DNA, 39 bp, Homo sapiens
tctggaggttcaggcggaaattttatgctgactcagccc

SEQ ID NO: 75 - DNA, 42 bp, Homo sapiens
tctggaggttcaggcggacagactgtggtgactcaggagccc

SEQ ID NO: 76 - DNA, 42 bp, Homo sapiens
tctggaggttcaggcggacaggctgtggtgactcaggagccc

SEQ ID NO: 77 - DNA, 41 bp, Homo sapiens
tctggaggttcaggcggacagactgtggtgacccaggagcc

SEQ ID NO: 78 - DNA, 41 bp, Homo sapiens
tctggaggttcaggcggacagcctgtgctgactcagccacc

SEQ ID NO: 79 - DNA, 42 bp, Homo sapiens
tctggaggttcaggcggacaggcagggctgactcagccaccc

SEQ ID NO: 80 - DNA, 48 bp, Homo sapiens
tctggaggttcaggcggacagtacgaattgactcagcctgcccctgcc

SEQ ID NO: 81 - DNA, 40 bp, Homo sapiens
tctggaggttcaggcggacagagcgaattgactcagcccc

SEQ ID NO: 82 - DNA, 39 bp, Homo sapiens
tctggaggttcaggcggattgactcagtcaccctcattg

SEQ ID NO: 83 - DNA, 39 bp, Homo sapiens
tctggaggttcaggcggattgactcagccaccctcagtg

SEQ ID NO: 84 - DNA, 41 bp, Homo sapiens
tctggaggttcaggcggaracatccagatgacccagtctcc

SEQ ID NO: 85 - DNA, 41 bp, Homo sapiens
tctggaggttcaggcggagmcatccagttgacccagtctcc

SEQ ID NO: 86 - DNA, 41 bp, Homo sapiens
tctggaggttcaggcggagccatcyggatgacccagtctcc

SEQ ID NO: 87 - DNA, 41 bp, Homo sapiens
tctggaggttcaggcggagccatccagatgacccagtctcc

SEQ ID NO: 88 - DNA, 45 bp, Homo sapiens
tctggaggttcaggcggagatattgtgatgacccagactccactc

SEQ ID NO: 89 - DNA, 45 bp, Homo sapiens
tctggaggttcaggcggagatgttgtgatgactcagtctccactc

SEQ ID NO: 90 - DNA, 41 bp, Homo sapiens
tctggaggttcaggcggagaaattgtgttgacrcagtctcc

SEQ ID NO: 91 - DNA, 41 bp, Homo sapiens
tctggaggttcaggcggagaaatagtgatgacgcagtctcc

SEQ ID NO: 92 - DNA, 41 bp, Homo sapiens
tctggaggttcaggcggagaaattgtaatgacgcagtctcc

SEQ ID NO: 93 - DNA, 41 bp, Homo sapiens
tctggaggttcaggcggagacatcgtgatgacccagtctcc

SEQ ID NO: 94 - DNA, 41 bp, Homo sapiens
tctggaggttcaggcggagaaacgacactcacgcagtctcc

SEQ ID NO: 95 - DNA, 41 bp, Homo sapiens
tctggaggttcaggcggagaaattgtgctgactcagtctcc

SEQ ID NO: 96 - DNA, 41 bp, Homo sapiens
tctggaggttcaggcggagatgttgtgatgacacagtctcc

SEQ ID NO: 97 - DNA, 42 bp, Homo sapiens
gcctgaacctccagaaccaagtttgatttccaccttggtccc

SEQ ID NO: 98 - DNA, 42 bp, Homo sapiens
gcctgaacctccagaaccaagtttgatctccagcttggtccc

SEQ ID NO: 99 - DNA, 42 bp, Homo sapiens
gcctgaacctccagaaccaagtttgatatccactttggtccc

SEQ ID NO: 100 - DNA, 42 bp, Homo sapiens
gcctgaacctccagaaccaagtttgatctccaccttggtccc

SEQ ID NO: 101 - DNA, 42 bp, Homo sapiens
gcctgaacctccagaaccaagtttaatctccagtcgtgtccc

SEQ ID NO: 102 - DNA, 42 bp, Homo sapiens
gcctgaacctccagaaccaagtctaagctccagtcgtgtccc

SEQ ID NO: 103 - DNA, 42 bp, Homo sapiens
gcctgaacctccagaaccaagcttgatttccaccttggtccc

SEQ ID NO: 104 - DNA, 42 bp, Homo sapiens
gcctgaacctccagaaccaactaggacggtgaccttggtccc

SEQ ID NO: 105 - DNA, 42 bp, Homo sapiens
gcctgaacctccagaaccaactaggacggtcagcttggtccc

SEQ ID NO: 106 - DNA, 42 bp, Homo sapiens
gcctgaacctccagaaccaacgaggacggtcagctgggtgcc

SEQ ID NO: 107 - DNA, 42 bp, Homo sapiens
gcctgaacctccagaaccaaccaggacggtgacctgggtccc

SEQ ID NO: 108 - DNA, 45 bp, Homo sapiens
gcctgaacctccagaaccaacgcttgagacggtgaccagggtgcc

SEQ ID NO: 109 - DNA, 45 bp, Homo sapiens
gcctgaacctccagaaccaacgcttgagacagtgaccagggtgcc

SEQ ID NO: 110 - DNA, 45 bp, Homo sapiens
gcctgaacctccagaaccaacgcttgagacggtgaccattgtccc

SEQ ID NO: 111 - DNA, 45 bp, Homo sapiens
gcctgaacctccagaaccaacgcttgagacggtgaccagggttcc

SEQ ID NO: 112 - DNA, 45 bp, Homo sapiens
gcctgaacctccagaaccaacgcttgagacggtgaccgtggtccc

SEQ ID NO: 113 - DNA, 45 bp, Homo sapiens
gcctgaacctccagaaccaacgcttgagacggtgaccagggtccc

SEQ ID NO: 114 - DNA, 59 bp, Homo sapiens
accaccgccaccagaaccaccaccaccagagccgccaccgcctgaacctccagaaccaa

SEQ ID NO: 115 - DNA, 57 bp, Homo sapiens
ggtggcggctctggtggtggtggttctggtggcggtggttctggaggttcaggcgga

SEQ ID NO: 116 - DNA, 59 bp, Homo sapiens
accgccaccgccagaaccgcccccgccagaaccgccgccgcctgaacctccagaaccaa

SEQ ID NO: 117 - DNA, 57 bp, Homo sapiens
ggcggcggttctggcgggggcggttctggcggtggcggttctggaggttcaggcgga

SEQ ID NO: 118 - DNA, 51 bp, Homo sapiens
gcatctatgttcgttttttctattgctacaaacgcgtatgcaagtgcacag

SEQ ID NO: 119 - DNA, 51 bp, Homo sapiens
gatcagcttttgttcggatccagcggccgaagcatcagcccgagcggccgc